(19) United States
(12) Patent Application Publication
(10) Pub. No.: US 2002/0143047 A1
(43) Pub. Date: Oct. 3, 2002

(54) Methods for treating indomethacin responsive headaches

(76) Inventors: Bradley S. Galer, West Chester, PA (US); Lawrence Newman, Goldens Bridge, NY (US)

Correspondence Address: Bret E. Field, BOZICEVIC, FIELD & FRANCIS LLP, Suite 200, 200 Middlefield Road, Menlo Park, CA, 94025, US

(21) Appl. No.: 09/755,592

(22) Filed: Jan. 5, 2001

(51) Int. Cl. A61K 31/405

(52) U.S. Cl. 514/420

(57) ABSTRACT

Methods and compositions, e.g., kits, are provided for treating a host suffering from an indomethacin responsive headache syndrome. In the subject methods, a topical indomethacin formulation is applied to a keratinized skin site proximal to the pain associated with the headache syndrome, e.g. a keratinized skin surface of the head, such as the forehead, temple, etc. Practice of the subject methods results in at least a reduction in the intensity of the pain associated with the syndrome. The subject methods and compositions find use in the treatment of a variety of indomethacin responsive headache syndromes, including syndromes that are absolutely responsive to indomethacin therapy and syndromes that are variably responsive to indomethacin therapy.

### TECHNICAL FIELD

The technical field of the invention is indomethacin-responsive headaches and the treatment thereof.

### BACKGROUND OF THE INVENTION

The indomethacin-responsive headaches are a group of uncommon primary headache disorders that, by accepted definition of the International Headache Society Diagnostic Criteria, only demonstrate a prompt remission following therapy with indomethacin. The indomethacin responsive headaches can be further separated into two subcategories, based upon clinical features and degree of responsiveness to indomethacin.

Those syndromes that exhibit an absolute response to therapy include the paroxysmal hemicranias and hemicrania continua. Syndromes exhibiting a variable response to therapy include idiopathic stabbing headaches (also called ice-pick headaches), and the benign forms of cough headache, exertional headache, and headaches associated with sexual activity. Unlike other primary headache disorders such as migraine, tension-type and cluster headaches, the indomethacin responsive headache conditions are resistant to treatment with all other classes of therapeutic agents.

Although indomethacin treatment will prevent this class of headaches, treatment needs to be maintained indefinitely, as prematurely discontinuing therapy or even delaying the dosage will result in the prompt recurrence of pain. Indomethacin, like aspirin and other non-steroidal anti-inflammatory agents (NSAIDS), is a cyclo-oxygenase inhibitor that is administered systemically. It is still unknown why this specific NSAID, indomethacin, is unique in resolving the pain of these headaches; other NSAIDS do not alleviate the pain associated with these headaches.

There is an acknowledged understanding by those of skill in the art that indomethacin must be administered systemically in order to be effective in the treatment of indomethacin responsive headaches. Currently, only systemically active (oral and rectal) formulations of indomethacin have been described and utilized for the treatment of indomethacin responsive headaches.

Oral and rectal administration of indomethacin frequently results in gastritis, i.e., stomach upset. Prolonged oral and rectal administration may induce gastric irritation and gastrointestinal ulceration. Long-term use may be associated with renal damage and increases in bleeding time and thus can cause serious adverse bodily reactions. The medication cannot be systemically administered to patients concurrently using aspirin-containing preparations, NSAIDS, warfarin, and certain anti-hypertensive agents. The use of indomethacin should be avoided in patients with active ulcer disease or history of ulcer disease, renal disorders and NSAID or aspirin allergies. Therefore, there are patients with indomethacin responsive headaches who cannot receive the appropnate treatment wince the only currently available systemic formulations are contra-indicated.

In view of the above problems with systemic administration of indomethacin in the treatment of indomethacin responsive headaches, there is an acknowledged need in the art for the development of new treatment modalities for this category of headaches that overcomes the above disadvantages experienced with current treatment modalities.

Relevant Literature

References of interest that discuss indomethacin responsive headache syndromes include: Newman et al., Neurology (1994) 44: 2111-4; Newman et al., Headache (1993) 33: 195-197; Newman et al., Headache (1992) 32:237-8; Newman et al., Neurology (1992) 42:964-6; Newman et al., Headache (1992) 32:75-76; Sands et al., Med. Clin. North. Am. (1991) 75: 733-747. Topical formulations of NSAIDS include those described in: U.S. Pat. Nos. 4,670,254; 4,710,497; 4,740,374; 4,777,046; 4,956,171; 4,999,379; 5,036,100; 5,204,119; 5,373,022; 5,374,661; 5,429,590; 5,695,779; 5,814,599; and EPB 0574255, the disclosures of which are herein incorporated by reference.

### SUMMARY OF THE INVENTION

Methods and compositions (e.g., kits) are provided for treating a host suffering from an indomethacin responsive headache syndrome. In the subject methods, a topical indomethacin formulation is applied to a keratinized skin site proximal to the pain associated with the headache syndrome, e.g., a keratinized skin surface of the head, such as the forehead, temple, orbital region, supraorbital region, etc. Practice of the subject methods results in at least a reduction in the intensity of the pain associated with the headache syndrome. The subject methods and compositions find use in the treatment of a variety of indomethacin responsive headache syndromes, including syndromes that are absolutely responsive to indomethacin therapy and syndromes that are variably responsive to indomethacin therapy.

### DESCRIPTION OF THE SPECIFIC EMBODIMENTS

Methods and compositions, e.g., kits, are provided for treating a host suffering from an indomethacin responsive headache syndrome. In the subject methods, a topical indomethacin formulation is applied to a keratinized skin site proximal to the pain associated with the headache syndrome, e.g., a keratinized skin surface of the head. Practice of the subject methods results in at least a reduction in the intensity of the pain associated with the syndrome. In further describing the subject invention, the methods will be described first followed by a review of kits for use in practicing the subject methods.

Before the subject invention is further described, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this invention belongs.

Methods

As summarized above, the subject invention provides a method for treating a host suffering from an indomethacin responsive headache syndrome. By indomethacin responsive headache syndrome is meant a headache syndrome that is resistant to treatment with all types of therapies except for indomethacin, and only demonstrates prompt remission following therapy with indomethacin. Indomethacin responsive headaches amenable to treatment according to the subject methods include both syndromes that are absolutely responsive to indomethacin treatment, e.g., paroxysmal hemicranias and hemicrania continuas, and syndromes that are variably responsive to indomethacin therapy, e.g., stabbing headaches, benign cough headaches, exertional headaches and headaches associated with sexual activity, etc.

As indicated above, topical indomethacin formulations are employed in the subject methods. In the broadest sense, any convenient topical formulation that provides for the requisite penetration of the indomethacin in the formulation through the keratinized skin surface and to the target area, e.g., nerves, tissue and vessels at which the headache pain originates, of the host may be employed. The topical formulation may be a gel, lotion, cream, patch, hydrogel patch, aerosol or some other topical formulation. The amount of indomethacin present in the topical formulation is one that is effective for at least reducing the pain associated with the syndrome being treated, and generally ranges from about 0.05 to 50% w/w, usually from about 0.1 to 40% w/w and more usually from about 0.1 to 25% w/w.

In many embodiments, the topical formulation is a patch formulation. Generally, the topical formulations include from about 0.05 to 30% w/w, usually from about 0.05 to 25% w/w and more usually from about 0.1 to 20% w/w of indomethacin. Other components of the patch compositions that may be present include polymers, adhesive resins, solubilizers, thickeners, and the like.

The topical composition may also contain other physiologically acceptable excipients or other minor additives, particularly associated with organoleptic properties, such as fragrances, dyes, emulsifiers, buffers, cooling agents (e.g. menthol), antibiotics, stabilizers or the like. The excipients and minor additives will be present in conventional amounts ranging from about 0.001% to 5%, more commonly 0.001-2%, by weight, usually not exceeding a total of 10% by weight.

In a particular type of patch formulations of interest, in addition to indomethacin present in the above ranges, the patch formulation further includes: (a) styrene-isoprene-styrene block copolymer, typically ranging from about 5 to 50% w/w and usually from about 10 to 30% w/w; (b) an adhesive resin, such as hydrogenated rosin glycerin ester, alicyclic saturated hydrocarbon resin, terpene resin, etc., typically ranging from about 5 to 50% w/w and usually from about 20 to 50% w/w; (c) polybutene, typically ranging from about 5 to 20% w/w and usually from about 7 to 15% w/w; (d) dibutylhydroxytoluene, typically ranging from about 0.1 to 5% w/w and usually from about 0.5 to 2% w/w; (e) and at least one solubilizer selected from the group consisting of chrotamitone, polyethylene glycol and N-methyl-2-pyrolidone, typically ranging from about 0.1 to 20% w/w. Specific indomethacin topical patch formulations are disclosed in the Experimental Section, supra.

In practicing the subject methods, the topical indomethacin formulation is applied to a keratinized skin surface of the host in a manner sufficient to provide for penetration of an affective amount of the indomethacin to the nerves causing the pain associated with the syndrome being treated. In other words, the topical indomethacin formulation is applied to the keratinized skin surface of the host at a region sufficiently proximal to the pain origin to provide for sufficient penetration of the indomethacin through the keratinized skin surface and to pain origin. Typically, the topical formulation is applied to a keratinized skin surface of the head, where head keratinized skin surfaces of interest include the forehead and regions thereof, the temples, the supraorbital and occipital regions, and the like. The topical formulation can be applied to one or more distinct regions, depending on the pain origin.

The amount of composition applied is one that is sufficient to provide for the desired reduction in pain intensity. The exact amount of topical composition that is applied may be determined empirically. For solutions, dispersions, gels, lotions, creams and the like, the composition will be spread over the region and a covering optionally applied thereto. For patches, an appropriate sized patch will be placed over the region comprising the skin site. Generally, the area of skin that is covered with topical formulations is at least about 8 cm², usually at least about 16 cm² and more usually at least about 25 cm² and may be as great as 40 cm² or greater, but typically does not exceed about 50 cm² and usually does not exceed about 60 cm².

The formulation is maintained in place for a period of time sufficient for the desired amelioration in symptoms to occur. Generally, the formulation is maintained in place for at least about 4 hr, usually at least about 6 hr and more usually for at least about 8 hr, where the formulation may be maintained in place for as long as 12 hr or longer.

Upon application of the topical composition, the indomethacin present therein penetrates the surface of the skin to reach the pain origin and reduce at least one symptom associated with the syndrome being treated. As such, application and maintenance of the topical indomethacin formulation as described above results in at least an amelioration of at least one symptom associated with the syndrome being treated. By “at least an amelioration” is meant at least a reduction in the magnitude of the symptom, including a complete cessation or removal of the symptom.

Typically, the symptom ameliorated by the subject methods is pain associated with the syndrome being treated. In many embodiments, the intensity of pain associated with the syndrome is at least reduced, if not eliminated. Reduction in pain intensity is evaluated according to the protocol described at pages 161-176, and particularly page 172, of “Guidelines for Controlled Trials of Drugs in Cluster Headache,” published by the International Headache Society Committee on Clinical Trials in Cluster Headache. The magnitude of reduction in pain intensity is generally at least about 10%, usually at least about 25% and more usually at least about 50%, where magnitude of reduction may be as high as 75%, 80%, 95% or higher, including a complete cessation of pain, as determined by the assay described above.

A variety of hosts are treatable according to the subject methods. Generally such hosts are “mammals” or “mammalian,” where these terms are used broadly to describe organisms which are within the class mammalia. Of particular interest is the treatment of primates with the subject methods, (e.g., humans, chimpanzees, and monkeys), where the subject methods are particularly suited for use in the treatment of humans suffering from indomethacin responsive headache syndromes, as described above.

Kits

Kits with indomethacin topical formulations used in the subject methods, are provided. Conveniently, the topical formulations may be provided in a unit dosage format, which formats are known in the art. In such kits, in addition to the containers containing the formulation, e.g. unit doses, is an informational package insert describing the use of the subject formulations in the methods of the subject invention, i.e. instructions for using the subject unit doses to treat indomethacin responsive headache syndromes via topical administration of an indomethacin formulations. Theses instructions may be present on one or more of the packaging, a package insert, and the like.

The following examples are offered by way of illustration and not by way of limitation.

### Experimental

I. Topical Formulation

| Ingredients | Amount (g) | (%) |
|---|---|---|
| Indomethacin | 0.07 | (5) |
| Styrene-isoprene-styrene block polymer | 0.21 | (15) |
| Hydrogenated rosin glycerin ester | 0.378 | (27) |
| Polybutene | 0.14 | (10) |
| Dibutylhydroxytoluene | 0.028 | (2) |
| I-Menthol | 0.007 | (1) |
| Perfume | Slight | |
| Liquid paraffin | Sufficient | |
| Total | 1.4 | |

Formulation A

II. Clinical Results

The topical formulation described in I, above, was employed to treat a male patient suffering from hemicrania continua and ulceritive colitis. The patient suffered from a six-year history of constant right-sided hemicranial pain of mild to moderate severity. Superimposed exacerbations of more intense pain occurred twice weekly and would last up to twelve hours. These painful exacerbations were associated with ipsilateral ptosis, conjunctival injection and lacrimation. Prior treatment with oral indomethacin, 25 mg, three times daily resulted in complete resolution of pain but was associated with significant worsening of the patient's ulcerative colitis. Decreasing the dosage did not abolish the headache syndrome and continued to aggravate the gastrointestinal symptoms so that indomethacin therapy had to be discontinued.

The patient was then treated with the topical application of indomethacin described in I, above, by applying the topical application to the site of maximal pain, i.e. the right forehead and temple. Application resulted in approximately 90% reduction in the daily pain within 15 minutes following the application (as determined according to the protocol described at pages 161-176, and particularly page 172, of “Guidelines for Controlled Trials of Drugs in Cluster Headache,” published by the International Headache Society Committee on Clinical Trials in Cluster Headache) and was maintained for approximately 12 hours. The patient was then instructed to reapply the patch twice daily, at twelve hour intervals. This procedure resulted in sustained pain relief to approximately 90% and prevented the painful exacerbations. The patient reported no adverse side-effects, specifically there were no associated flare-ups of ulceritive colitis. The patient has been maintained on this therapy for two months without evidence of tachyphlaxis or adverse events. This was a favorable result compared with other forms of treatment that the patient had previously employed, whereas despite appropriate therapy, the patient's other medical condition worsened in response to oral administration of indomethacin.

III. Alternative Topical Indomethacin Formulations

B. Alternative Tape Formulations

| | |
|---|---|
| Styrene-isoprene-styrene block polymer | 15% |
| Adhesive resin (hydrogenated rosin glycerin ester) | 30% |
| Liquid paraffin | 30% |
| Polybutene | 10% |
| Dibutylhydroxytoluene | 2% |
| Crotamitone | 12% |
| Indomethacin | 1% |
| | 100% |
| Formulation B.1 | |
| Styrene-isoprene-styrene block polymer | 20% |
| Adhesive resin (alicyclic saturated hydrocarbon resin) | 35% |
| Liquid paraffin | 20% |
| Polybutene | 10% |
| Dibutylhydroxytoluene | 2% |
| Polyethylene glycol | 10% |
| Indomethacin | 3% |
| | 100% |
| Formulation B.2 | |
| Styrene-isoprene-styrene block polymer | 30% |
| Adhesive resin (terpene resin) | 40% |
| Liquid paraffin | 10% |
| Polybutene | 8% |
| Dibutylhydroxytoluene | 2% |
| N-methyl-2-pyrolidone | 5% |
| Indomethacin | 5% |
| | 100% |
| Formulation B.3 | |
| Styrene-isoprene-styrene block polymer | 15% |
| Adhesive resin (hydrogenated rosin glycerin ester) | 30% |
| Liquid paraffin | 23% |
| Polybutene | 10% |
| Dibutylhydroxytoluene | 2% |
| N-methyl-2-pyrolidone | 5% |
| Polyethylene glycol 2000 | 5% |
| Indomethacin | 10% |
| | 100 |
| Formulation B.4 | |
| Styrene-isoprene-styrene block polymer | 13% |
| Adhesive resin (hydrogenated rosin glycerin ester) | 27% |
| Liquid paraffin | 22% |
| Polybutene | 10% |
| Dibutylhydroxytoluene | 2% |
| N-methyl-2-pyrolidone | 7% |
| Crotamitone | 7% |
| Indomethacin | 12% |
| | 100% |
| Formulation B.5 | |
| Styrene-isoprene-styrene block polymer | 12% |
| Adhesive resin (hydrogenated rosin glycerin ester) | 23% |
| Liquid paraffin | 25% |
| Polybutene | 8% |
| Dibutyihydroxytoluene | 2% |
| N-methyl-2-pyrolidone | 5% |
| Crotamitone | 5% |
| Polyethylene glycol | 5% |
| Indomethacin | 15% |
| | 100% |
| Formulation 8.6 | |

C. Alternative Non-Tape Formulations

| Ingredients | % weight/weight | |
|---|---|---|
| Indomethacin | 0.75 | |
| Diisopropyl Adipate | 5.0 | |
| Isopropyl Myristate | 3.0 | |
| Glycerin | 2.0 | |
| Polyethylene Alkyl Ether | 3.0 | |
| Ethanol | 45.0 | |
| Purified Water | Total 100 | mL |
| Formulation C | | |
| Indomethacin | 1 | |
| Medium Chain Fatty Acid Triglyceride | 20 | |
| Diisopropyl Adipate | 5 | |
| Propylene Glycol | 12 | |
| Polyoxyethylene Sorbitan Monostearate | 6 | |
| Sorbitan Monostearate | 3 | |
| Glycerin Monostearate | 8 | |
| Purified Water | Total 100 | g |
| Formulation D | | |
| Indomethacin | 0.5 | |
| Polyethylene Glycol Monostearate | 5 | |
| Diisopropyl Adipate | 3 | |
| 1,3-Butylene Glycol | 8 | |
| Polyvinyl Pyrrolidone | 0.5 | |
| Carboxyvinyl Polymer | 1.5 | |
| Diisopropanolamine | Sufficient | |
| Ethanol | 30 | |
| Purified Water | Total 100 | g |
| Formulation E | | |
| Indomethacin | 0.5 | |
| Polyoxyethylene Sorbitan Monooleate | 1 | |
| Propylene Glycol | 5 | |
| Polyacrylic Acid | 7 | |
| Sodium Polyacrylate | 6 | |
| Anhydrous Silicic Acid | 1 | |
| Tartaric Acid | 0.5 | |
| Aluminium Glycinate | Sufficient | |
| Aluminium Hydroxide | Sufficient | |
| Purified Water | Total 100 | g |
| Formulation F | | |
| Indomethacin | 0.4 | |
| Polyoxyethylene Sorbitan Tristearate | 1.2 | |
| Diisopropyl Adipate | 2 | |
| 1,3-Butylene Glycol | 1.2 | |
| Ethanol | 20 | |
| Purified Water | 12.2 | |
| N-Pentane | 10 | |
| Liquified Petroleum Gas | 3 | |
| Dimethyl Ether | 47 | |
| Formulation G | | |

It is evident from the above results and discussion that a novel and effective method of treating headache pain in a host is provided. The nature of the topical applications employed provides for rapid penetration of the skin surface by indomethacin. As a result, the patient experiences pain relief shortly after application that is maintained for approximately 12 hours. Furthermore since the composition is topical, the method is well tolerated by patients.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method for at least reducing pain intensity associated with an indomethacin responsive headache syndrome in a host, said method comprising:
topically applying an effective amount of a topical indomethacin formulation to a keratinized skin surface of said subject sufficiently proximal pain associated with said syndrome to at least reduce said intensity.

2. The method according to claim 1, wherein said syndrome is a syndrome that is absolutely response to indomethacin therapy.

3. The method according to claim 2, wherein said absolutely responsive syndrome is selected from the group consisting of paroxysmal hemicranias and hemicrania continua.

4. The method according to claim 1, wherein said syndrome is a syndrome that exhibits variable response to indomethacin therapy.

5. The method according to claim 4, wherein said syndrome is selected from the group consisting of stabbing headaches, benign cough headaches, exertional headaches and headaches associated with sexual activity.

6. The method according to claim 1, where said topical formulation is in the form of a patch.

7. The method according to claim 1, wherein said topical formulation is in the form of a lotion.

8. The method according to claim 1, wherein said topical formulation is in the form of a gel.

9. The method according to claim 1, wherein said keratinized skin surface is present on the head of said host.

10. The method according to claim 9, wherein said keratinized skin surface includes at least part of the forehead of said host.

11. The method according to claim 1, wherein said host is a mammal.

12. The method according to claim 11, wherein said mammal is a human.

13. A kit for use in treating a subject suffering from an indomethacin responsive headache syndrome, said kit comprising:
a topical indomethacin formulation; and
instructions for practicing the method according to claim 1.

14. The kit according to claim 13, wherein said formulation is a patch.

15. The kit according to claim 14, wherein said patch comprises from about 0.1 to 25% w/w indomethacin.

16. The kit according to claim 15, wherein said patch comprises from about 0.1 to 20% w/w indomethacin.
